(19) United States
(12) Patent Application Publication
(10) Pub. No.: US 2020/0131102 A1
(43) Pub. Date: Apr. 30, 2020

(54) Process for Catalytic Oxidative Conversion of Methane to Ethylene in the Presence of Chlorine Intermediates

(71) Applicant: Sabic Global Technologies, B.V., BERGEN OP ZOOM (NL)

(72) Inventor: Aghaddin MAMEDOV, Sugar Land, TX (US)

(21) Appl. No.: 16/483,689

(22) PCT Filed: Feb. 5, 2018
(86) PCT No.: PCT/US2018/016833

§ 371 Date: Aug. 5, 2019

Related U.S. Application Data
(60) Provisional application No. 62/455,766, filed on Feb. 7, 2017.

(51) Int. Cl. C07C 11/04; C07C 2/84

(52) U.S. Cl. CPC C07C 11/04; C07C 2523/04; C07C 2/84

(57) ABSTRACT

A process for producing ethylene comprising (a) contacting a reactant mixture with an oxidative coupling of methane (OCM) catalyst in the presence of a chlorine intermediate precursor in a reactor to yield a product mixture, wherein the reactant mixture comprises methane and oxygen, wherein the product mixture comprises ethylene, ethane, and unreacted methane, and wherein the OCM catalyst comprises an alkali metal, an alkaline earth metal, or both; and (b) recovering at least a portion of the ethylene from the product mixture. Yielding the product mixture in step (a) further comprises (i) allowing a first portion of the reactant mixture to react via an OCM reaction, (ii) allowing at least a portion of the chlorine intermediate precursor to generate a chlorine intermediate, and (iii) allowing a second portion of the reactant mixture to react via the chlorine intermediate.

### CROSS REFERENCE TO RELATED APPLICATIONS

This application is a filing under 35 U.S.C. 371 of International Application No. PCT/US2017/016833 filed Feb. 5, 2018, entitled “A Process for Catalytic Oxidative Conversion of Methane to Ethylene in the Presence of Chlorine Intermediates” which claims priority to U.S. Provisional Application No. 62/455,766 filed Feb. 7, 2017, which applications are incorporated by reference herein in their entirety.

### TECHNICAL FIELD

The present disclosure relates to methods of producing hydrocarbons, more specifically methods of producing olefins, such as ethylene, by oxidative coupling of methane in the presence of chlorine radicals.

### BACKGROUND

Hydrocarbons, and specifically olefins such as ethylene (C₂H₄), are typically building blocks used to produce a wide range of products, for example, break-resistant containers and packaging materials. Currently, for industrial scale applications, ethylene is produced by heating natural gas condensates and petroleum distillates, which include ethane and higher hydrocarbons, and the produced ethylene is separated from a product mixture by using gas separation processes.

Oxidative coupling of the methane (OCM) has been the target of intense scientific and commercial interest for more than thirty years due to the tremendous potential of such technology to reduce costs, energy, and environmental emissions in the production of C₂H₄. As an overall reaction, in the OCM, CH₄ and O₂ react exothermically over a catalyst to produce C₂H₄, water (H₂O) and heat.

Ethylene can be produced by OCM as represented by Equations (I) and (II):

2CH₄+O₂→C₂H₄+2H₂O ΔH=−67 kcal/mol (I)

2CH₄+½O₂→C₂H₆+H₂O ΔH=−42 kcal/mol (II)

Oxidative conversion of methane to ethylene is exothermic. Excess heat produced from these reactions (Equations (I) and (II)) can push conversion of methane to carbon monoxide and carbon dioxide rather than the desired C₂ hydrocarbon product (e.g., ethylene):

CH₄+1.5O₂→CO+2H₂O ΔH=−124 kcal/mol (III)

CH₄+2O₂→CO₂+2H₂O ΔH=−192 kcal/mol (IV)

The excess heat from the reactions in Equations (III) and (IV) further exasperate this situation, thereby substantially reducing the selectivity of ethylene production when compared with carbon monoxide and carbon dioxide production.

Additionally, while the overall OCM is exothermic, catalysts are used to overcome the endothermic nature of the C—H bond breakage. The endothermic nature of the bond breakage is due to the chemical stability of methane, which is a chemically stable molecule owing to the presence of its four strong tetrahedral C—H bonds (435 kJ/mol). When catalysts are used in the OCM, the exothermic reaction can lead to a large increase in catalyst bed temperature and uncontrolled heat excursions that can lead to catalyst deactivation and a further decrease in ethylene selectivity. Furthermore, the produced ethylene is highly reactive and can form unwanted and thermodynamically favored deep oxidation products.

An overall yield of desired C₂ hydrocarbons is reduced by non-selective reactions of methyl radicals with oxygen on the catalyst surface and/or in the gas phase, which produce (undesirable) carbon monoxide and carbon dioxide. Some of the best reported OCM outcomes encompass a ˜20% conversion of methane and ˜80% selectivity to desired C₂ hydrocarbons.

Alternatively, methane can be converted to methyl chloride via oxidative conversion, and methyl chloride can be further converted to olefins such as ethylene in the presence of zeolite catalysts. However, zeolite catalysts employed in such reactions suffer from very fast deactivation.

Thus, there is an ongoing need for the development of OCM processes that can increase the production of ethylene, while reducing catalyst deactivation.

### BRIEF SUMMARY

Disclosed herein is a process for producing ethylene comprising (a) contacting a reactant mixture with an oxidative coupling of methane (OCM) catalyst in the presence of a chlorine intermediate precursor in a reactor to yield a product mixture, wherein the reactant mixture comprises methane and oxygen, wherein the product mixture comprises ethylene, ethane, and unreacted methane, and wherein the OCM catalyst comprises an alkali metal, an alkaline earth metal, or both, and (b) recovering at least a portion of the ethylene from the product mixture.

Also disclosed herein is a process for producing ethylene comprising (a) continuously feeding a reactant mixture to a reactor to yield a product mixture, wherein the reactor comprises an oxidative coupling of methane (OCM) catalyst, wherein the reactant mixture comprises methane, oxygen, and a chlorine radical precursor, wherein the chlorine radical precursor is present in the reactant mixture in an amount of from about 0.5 vol. % to about 3 vol. %, based on the total volume of the reactant mixture, wherein the product mixture comprises ethylene, ethane, and unreacted methane, wherein the OCM catalyst comprises (1) a redox agent in an amount of from about 1 wt. % to about 25 wt. %, based on the total weight of the OCM catalyst, and (2) an alkali metal, an alkaline earth metal, or both, in an amount of less than about 3 wt. %, based on the total weight of the OCM catalyst, and (b) recovering at least a portion of the ethylene from the product mixture.

Further disclosed herein is a process for producing ethylene comprising (a) continuously feeding a reactant mixture and a chlorine radical precursor for an activation time period to a reactor comprising an oxidative coupling of methane (OCM) catalyst to activate the OCM catalyst and to yield a first product mixture, wherein the chlorine radical precursor is introduced to the reactor in an amount of from about 2 vol. % to about 5 vol. %, based on the total volume of the reactant mixture, wherein the reactant mixture comprises methane and oxygen, wherein the first product mixture comprises ethylene, ethane, and unreacted methane, wherein the OCM catalyst comprises (1) a redox agent in an amount of from about 1 wt. % to about 25 wt. %, based on the total weight of the OCM catalyst, and (2) an alkali metal, an alkaline earth metal, or both, in an amount of equal to or greater than about 3 wt. %, based on the total weight of the OCM catalyst, (b) discontinuing the introduction of the chlorine radical precursor to the reactor while continuing to feed the reactant mixture to the reactor for a reaction time period to produce a second product mixture, wherein the second product mixture comprises ethylene, ethane, and unreacted methane, (c) repeating steps (a) and (b) as necessary to achieve a target methane conversion and/or a target ethylene selectivity, and (d) recovering at least a portion of the ethylene from the first product mixture and/or the second product mixture, wherein the first product mixture and/or the second product mixture are characterized by an ethylene to ethane molar ratio of equal to or greater than about 8:1.

### DETAILED DESCRIPTION

Disclosed herein are processes for producing ethylene comprising (a) contacting a reactant mixture with an oxidative coupling of methane (OCM) catalyst in the presence of a chlorine intermediate precursor in a reactor to yield a product mixture, wherein the reactant mixture comprises methane and oxygen, wherein the product mixture comprises ethylene, ethane, and unreacted methane, and wherein the OCM catalyst comprises an alkali metal, an alkaline earth metal, or both; and (b) recovering at least a portion of the ethylene from the product mixture. In an aspect, the chlorine intermediate precursor is a chlorine radical precursor. In some aspects, the chlorine intermediate precursor can be introduced continuously to the reactor, wherein the OCM catalyst comprises the alkali metal, the alkaline earth metal, or both in an amount of less than about 3 wt. %, based on the total weight of the OCM catalyst. In other aspects, the chlorine intermediate precursor can be introduced discontinuously to the reactor, wherein the OCM catalyst comprises the alkali metal, the alkaline earth metal, or both in an amount of equal to or greater than about 3 wt. %, based on the total weight of the OCM catalyst.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an aspect,” “another aspect,” “other aspects,” “some aspects,” and so forth, means that a particular element (e.g., feature, structure, property, and/or characteristic) described in connection with the aspect is included in at least an aspect described herein, and may or may not be present in other aspects. In addition, it is to be understood that the described element(s) can be combined in any suitable manner in the various aspects.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group.

In an aspect, a process for producing ethylene as disclosed herein can comprise contacting a reactant mixture with an oxidative coupling of methane (OCM) catalyst in the presence of a chlorine intermediate precursor in a reactor to yield a product mixture, wherein the reactant mixture comprises methane (CH₄) and oxygen (O₂), wherein the product mixture comprises ethylene (C₂H₄), ethane (C₂H₆), and unreacted methane, and wherein the OCM catalyst comprises an alkali metal, an alkaline earth metal, or both. As will be appreciated by one of skill in the art, and with the help of this disclosure, the reactant mixture may comprise one or more reactive components (e.g., one or more hydrocarbons, such as methane; oxygen) and one or more inert components (e.g., a diluent, such as nitrogen, water, etc.), and the one or more reactive components of the reactant mixture may react in order to form one or more reaction products (e.g., C₂H₄, C₂H₆).

In an aspect, the reactor (e.g., OCM reactor) can comprise an adiabatic reactor, an autothermal reactor, a tubular reactor, a continuous flow reactor, and the like, or combinations thereof. In an aspect, the reactor can be characterized by a pressure of from about ambient pressure (e.g., atmospheric pressure) to about 500 psig, alternatively from about ambient pressure to about 200 psig, or alternatively from about ambient pressure to about 100 psig. In an aspect, the process for producing ethylene as disclosed herein can be carried out at ambient pressure.

The reactant mixture can comprise a hydrocarbon or mixtures of hydrocarbons, and oxygen. In some aspects, the hydrocarbon or mixtures of hydrocarbons can comprise natural gas (e.g., CH₄), liquefied petroleum gas comprising C₂-C₅ hydrocarbons, C₆₊ heavy hydrocarbons (e.g., C₆ to C₂₄ hydrocarbons, such as diesel fuel, jet fuel, gasoline, tars, kerosene, etc.), oxygenated hydrocarbons, biodiesel, alcohols, dimethyl ether, and the like, or combinations thereof. In an aspect, the reactant mixture can comprise CH₄ and O₂.

In some aspects, the reactant mixture can comprise C₂H₆, wherein the C₂H₆ can undergo conversion to C₂H₄ in the presence of an OCM catalyst and a chlorine intermediate precursor as disclosed herein.

The O₂ used in the reactant mixture can be oxygen gas (which may be obtained via a membrane separation process), technical oxygen (which may contain some air), air, oxygen enriched air, and the like, or combinations thereof.

The reactant mixture can further comprise a diluent. The diluent is inert with respect to the methane conversion reactions, e.g., the diluent does not participate in the methane conversion reactions. In an aspect, the diluent can comprise water, steam, nitrogen, inert gases (e.g., argon), and the like, or combinations thereof. In an aspect, the diluent can be present in the reactant mixture in an amount of from about 0.5% to about 80%, alternatively from about 5% to about 50%, or alternatively from about 10% to about 30%, based on the total volume of the reactant mixture.

In an aspect, the reactant mixture can be characterized by a CH₄/O₂ molar ratio of from about 2:1 to about 10:1, alternatively from about 3:1 to about 9:1, or alternatively from about 4:1 to about 8:1.

The OCM catalyst comprises an alkali metal, an alkaline earth metal, or both an alkali metal and an alkaline earth metal.

The alkali metal comprises sodium (Na), potassium (K), rubidium (Rb), cesium (Cs), or combinations thereof. In an aspect, the alkali metal is sodium (Na).

The alkaline earth metal comprises magnesium (Mg), calcium (Ca), strontium (Sr), barium (Ba), or combinations thereof. In an aspect, the alkaline earth metal is calcium (Ca).

In an aspect, the OCM catalyst comprises an alkali metal, an alkaline earth metal, or both in an amount of less than about 20 wt. %, alternatively less than about 15 wt. %, alternatively less than about 10 wt. %, alternatively less than about 5 wt. %, alternatively less than about 3 wt. %, alternatively from about 0.5 wt. % to about 3 wt. %, alternatively from about 1 wt. % to about 3 wt. %, alternatively from about 1.5 wt. % to about 2.5 wt. %, alternatively equal to or greater than about 0.5 wt. %, alternatively equal to or greater than about 1 wt. %, alternatively equal to or greater than about 1.5 wt. %, alternatively equal to or greater than about 2.5 wt. %, alternatively equal to or greater than about 3 wt. %, alternatively from about 0.5 wt. % to about 20 wt. %, alternatively from about 1 wt. % to about 20 wt. %, alternatively from about 1.5 wt. % to about 20 wt. %, alternatively from about 2 wt. % to about 20 wt. %, alternatively from about 2.5 wt. % to about 20 wt. %, alternatively from about 3 wt. % to about 20 wt. %, alternatively from about 5 wt. % to about 17.5 wt. %, alternatively from about 7.5 wt. % to about 17.5 wt. %, or alternatively from about 10 wt. % to about 15 wt. %, based on the total weight of the OCM catalyst.

The OCM catalyst can further comprise a redox agent. Nonlimiting examples of redox agents suitable for use in the OCM catalysts of the present disclosure include manganese (Mn), tin (Sn), bismuth (Bi), cerium (Ce), and the like, or combinations thereof. In an aspect, the redox agent is manganese (Mn).

In an aspect, the OCM catalyst can comprise the redox agent in an amount of from about 1 wt. % to about 25 wt. %, alternatively from about 1 wt. % to about 25 wt. %, alternatively from about 5 wt. % to about 22.5 wt. %, alternatively from about 10 wt. % to about 20 wt. %, or alternatively from about 15 wt. % to about 20 wt. %, based on the total weight of the OCM catalyst.

The OCM catalyst can comprise one or more oxides, such as basic oxides; mixtures of basic oxides; redox agents; redox agents with basic properties; mixtures of redox agents with basic properties; mixtures of redox agents with basic properties promoted with alkali metals and/or alkaline earth metals; rare earth metal oxides (e.g., oxides of rare earth elements); mixtures of rare earth metal oxides; mixtures of rare earth metal oxides promoted by alkali metals and/or alkaline earth metals; manganese; manganese compounds; lanthanum; lanthanum compounds; sodium; sodium compounds; cesium; cesium compounds; calcium; calcium compounds; and the like; or combinations thereof.

In an aspect, the OCM catalysts suitable for use in the present disclosure can be supported catalysts and/or unsupported catalysts. In some aspects, the supported catalysts can comprise a support, wherein the support can be catalytically active (e.g., the support can catalyze an OCM reaction). For example, the catalytically active support can comprise a metal oxide support, such as MgO. In other aspects, the supported catalysts can comprise a support, wherein the support can be catalytically inactive (e.g., the support cannot catalyze an OCM reaction), such as SiO₂. In yet other aspects, the supported catalysts can comprise a catalytically active support and a catalytically inactive support.

In some aspects, the support comprises an inorganic oxide, alpha, beta or theta alumina (Al₂O₃), activated Al₂O₃, silicon dioxide (SiO₂), titanium dioxide (TiO₂), magnesium oxide (MgO), calcium oxide (CaO), strontium oxide (SrO), zirconium oxide (ZrO₂), zinc oxide (ZnO), lithium aluminum oxide (LiAlO₂), magnesium aluminum oxide (MgAlO₄), manganese oxides (MnO, MnO₂, Mn₃O₄), lanthanum oxide (La₂O₃), activated carbon, silica gel, zeolites, activated clays, silicon carbide (SiC), diatomaceous earth, magnesia, aluminosilicates, calcium aluminate, carbonates, MgCO₃, CaCO₃, SrCO₃, BaCO₃, Y₂(CO₃)₃, La₂(CO₃)₃, and the like, or combinations thereof. In some aspects, the support can comprise MgO, Al₂O₃, SiO₂, ZrO₂, and the like, or combinations thereof.

In an aspect, the OCM catalysts suitable for use in the present disclosure can further comprises a support, wherein at least a portion of the OCM catalyst contacts, coats, is embedded in, is supported by, and/or is distributed throughout at least a portion of the support.

Nonlimiting examples of OCM catalysts suitable for use in the present disclosure include CeO₂, La₂O₃—CeO₂, Ca/CeO₂, Mn/Na₂WO₄, Li₂O, Na₂O, Cs₂O, WO₃, Mn₃O₄, CaO, MgO, SrO, BaO, CaO—MgO, CaO—BaO, Li/MgO, MnO, Ca—Mn—O/SiO₂, W₂O₃, SnO₂, Yb₂O₃, Sm₂O₃, MnO—W₂O₃, MnO—W₂O₃—Na₂O, MnO—W₂O₃—Li₂O, SrO/La₂O₃, La₂O₃, Ce₂O₃, La/MgO, La₂O₃—CeO₂—Na₂O, La₂O₃—CeO₂—CaO, Na₂O—MnO—WO₃—La₂O₃, La₂O₃—CeO₂—MnO—WO₃—SrO, Na—Mn—La₂O₃/Al₂O₃, Na—Mn/SiO₂, Na—Mn—O/SiO₂, Na₂WO₄-Mn/SiO₂, Na₂WO₄—Mn—O/SiO₂, Na/Mn/O, Na₂WO₄, Mn₂O₃/Na₂WO₄, Mn₃O₄/Na₂WO₄, MnW0₄/Na₂WO₄, MnWO₄/Na₂WO₄, Mn/WO₄, Na₂WO₄/Mn, Sr/Mn—Na₂WO₄, and the like, or combinations thereof.

In an aspect, yielding the product mixture can comprise allowing a first portion of the reactant mixture to react via an OCM reaction, in the presence of the OCM catalyst, as represented by equations (1) and (2):

2CH₄+O₂→C₂H₄+2H₂O (1)

2CH₄+½O₂→C₂H₆+H₂O (2)

Generally, in the OCM, CH₄ is first oxidatively converted into C₂H₆, and then into C₂H₄. CH₄ is activated heterogeneously on a catalyst surface (e.g., OCM catalyst), forming methyl free radicals (e.g., CH₃.), which then couple in a gas phase to form C₂H₆. C₂H₆ subsequently undergoes dehydrogenation to form C₂H₄.

The OCM reactions are generally accompanied by deep oxidation reactions, as represented by equations (3) and (4):

CH₄+1.5O₂→CO+2H₂O (3)

CH₄+2O₂→CO₂+2H₂O (4)

In some aspects, the chlorine intermediate precursor can be introduced to the reactor as part of the reactant mixture, e.g., the chlorine intermediate precursor can be added to the reactant mixture (e.g., methane and oxygen) and can be introduced to the reactor with the methane and oxygen via a common stream. In other aspects, the chlorine intermediate precursor can be introduced to the reactor via a stream other than the feed stream for methane and oxygen (e.g., separate stream).

In an aspect, yielding the product mixture can further comprise (i) allowing at least a portion of the chlorine intermediate precursor to generate a chlorine intermediate, and (ii) allowing a second portion of the reactant mixture to react via the chlorine intermediate.

In an aspect, the chlorine intermediate precursor can be a chlorine radical precursor, wherein the chlorine intermediate is a chlorine radical. The chlorine intermediate precursor can comprise hydrogen chloride (HCl), methyl chloride (CH₃Cl), methylene chloride (CH₂Cl₂), chloroform (CHCl₃), carbon tetrachloride (CCl₄), ethyl chloride (C₂H₅Cl), 1,2-dichloroethane (C₂H₄Cl₂), trichloroethylene (C₂HCl₃), and the like, or combinations thereof.

The stoichiometric reactions of methane conversion with the introduction of a chlorine intermediate precursor to the reactor can be described similar to the case of OCM reaction, as represented by equations (5)-(9) for various chlorine intermediate precursors:

2CH₄+O₂+(HCl)→C₂H₄+2H₂O (5)

2CH₄+O₂+(CH₃Cl)→C₂H₄+2H₂O (6)

2CH₄+O₂+(CH₂Cl₂)→C₂H₄+2H₂O (7)

2CH₄+O₂+(CCl₄)→C₂H₄+2H₂O (8)

2CH₄+O₂+(C₂H₅Cl)→C₂H₄+2H₂O (9)

Without wishing to be limited by theory, the chlorine intermediate precursors do not get consumed in reactions (5)-(9), due to a radical mechanism of chlorine participation in the reactions that can regenerate the chlorine intermediate precursors.

The chlorine intermediate precursor can be introduced to the reactor in an amount of from about 0.5 vol. % to about 5 vol. %, alternatively from about 0.5 vol. % to about 3 vol. %, alternatively from about 0.75 vol. % to about 3 vol. %, alternatively from about 1 vol. % to about 3 vol. %, alternatively from about 0.75 vol. % to about 4 vol. %, alternatively from about 1 vol. % to about 5 vol. %, alternatively from about 2 vol. % to about 5 vol. %, alternatively from about 2.5 vol. % to about 4.5 vol. %, or alternatively from about 3 vol. % to about 4 vol. %, based on the total volume of the reactant mixture. In some aspects, the chlorine intermediate precursor can be introduced continuously to the reactor. In other aspects, the chlorine intermediate precursor can be introduced discontinuously to the reactor.

In an aspect, the chlorine intermediate precursor can generate a chlorine intermediate via contacting at least a portion of the chlorine intermediate precursor with the OCM catalyst to form a chlorinated OCM catalyst, wherein at least a portion of the chlorinated OCM catalyst can generate the chlorine intermediate. The chlorine intermediate precursor can be contacted with the OCM catalyst either continuously or discontinuously, as disclosed herein. Without wishing to be limited by theory, the alkali metal and/or alkaline earth metal of the OCM catalyst reacts with the chlorine intermediate precursor that was introduced in a gas phase (e.g., with the reactant mixture) to the reactor and forms chlorides on a catalyst surface. As will be appreciated by one of skill in the art, and with the help of this disclosure, the more alkali metal and/or alkaline earth metal in the OCM catalyst, the more chlorides will be formed on the catalyst surface (e.g., the larger the chlorides surface coverage on the catalyst surface). For purposes of the disclosure herein, the term “chlorine intermediate precursor” refers to both a chlorine compound introduced in gas phase to the reactor, as well as a chloride or any other chlorine-containing compound adsorbed and/or formed on a surface of the OCM catalyst (e.g., chlorinated OCM catalyst), wherein such chloride or any other chlorine-containing compound adsorbed and/or formed on a surface of the OCM catalyst can further generate a chlorine intermediate, such as a chlorine radical. For purposes of the disclosure herein, the term “chlorinated OCM catalyst” refers to an OCM catalyst having a chloride or any other chlorine-containing compound adsorbed and/or formed on a surface of the OCM catalyst, wherein such chloride or any other chlorine-containing compound adsorbed and/or formed on a surface of the OCM catalyst (e.g., chlorinated OCM catalyst) can further generate a chlorine intermediate, such as a chlorine radical.

Without wishing to be limited by theory, the chloride or any other chlorine-containing compound adsorbed and/or formed on the OCM catalyst surface (e.g., chlorinated OCM catalyst) can further generate the chlorine intermediate (e.g., chlorine radical), for example via a redox agent, such as Mn. Redox agents can generally convert between oxide forms and chloride forms, which can lead to the formation of chlorine intermediates, thus promoting methane conversion reactions via chlorine intermediates. Further, and without wishing to be limited by theory, the chloride or any other chlorine-containing compound adsorbed and/or formed on the OCM catalyst surface can react with oxygen centers on the chlorinated OCM catalyst (e.g., on the catalyst surface) to generate the chlorine intermediate (e.g., chlorine radical), for example by reducing such oxygen centers on the OCM catalyst while oxidizing a chloride to a chlorine radical. Further, and without wishing to be limited by theory, chlorides or any other chlorine-containing compound adsorbed and/or formed on the chlorinated OCM catalyst surface can decrease the amount of oxygen available for deep oxidation reactions (e.g., by reducing oxygen centers), thereby minimizing deep oxidation reactions, for example deep oxidation reactions of methane to carbon dioxide.

For example, when the OCM catalyst comprises Mn, such as in the form of manganese oxides (e.g., MnO₂), and when the chlorine intermediate precursor comprises HCl, the generation of chlorine radicals (Cl.) can be represented by equations (10) and (11):

MnO₂+4HCl [symbol] MnCl₂+Cl₂+2H₂O (10)

Cl₂ [symbol] 2Cl. (11)

Without wishing to be limited by theory, the chlorine intermediate (e.g., chlorine radical, Cl.) can diffuse away from the catalyst surface (e.g., into the gas phase) and can initiate the formation of methyl radicals, ethyl radicals, and ultimately the formation of ethylene molecules. The chlorine intermediate (e.g., chlorine radical, Cl.) can re-generate the HCl while forming various alkyl radicals (e.g., methyl radicals (CH₃.), ethyl radicals (C₂H₅.), etc.), and such HCl can re-initiate the steps of forming the chlorine radical by interacting with the catalyst (e.g., for example according to reactions (10) and (11)); and/or in gas phase, for example as represented by equations (12)-(15):

CH₄+Cl. [symbol] CH₃.+HCl (12)

2CH₃. [symbol] C₂H₆ (13)

C₂H₆+Cl. [symbol] C₂H₅.+HCl (14)

C₂H₅. [symbol] C₂H₄+H. (15)

As disclosed herein, in the presence of chlorine intermediate precursors, (A) the OCM reactions (e.g., a first portion of the reactant mixture reacting via an OCM reaction, for example as represented by equations (1) and (2)) and (B) reactions via a chlorine intermediate (e.g., a second portion of the reactant mixture reacting via the chlorine intermediate, for example as represented by equations (12)-(15)) can occur simultaneously.

In an aspect, the chlorine intermediate precursor can be introduced continuously to the reactor. In such aspect, the OCM catalyst can comprise the alkali metal, the alkaline earth metal, or both in an amount of less than about 3 wt. %, alternatively from about 0.5 wt. % to about 3 wt. %, alternatively from about 1 wt. % to about 3 wt. %, or alternatively from about 1.5 wt. % to about 2.5 wt. %, based on the total weight of the OCM catalyst. Without wishing to be limited by theory, if the amount of the alkali metal, the alkaline earth metal, or both in the OCM catalyst is too high (e.g., equal to or greater than about 3 wt. %, based on the total weight of the OCM catalyst), then the entire surface of the catalyst would be covered by chlorides and/or other chlorine-containing compounds when continuously introducing the chlorine intermediate precursor to the reactor, thus hindering the ability of the OCM catalyst to promote other reactions, such as OCM reactions (e.g., equations (1) an (2)). Further, and without wishing to be limited by theory, when the catalyst surface is almost fully covered in chlorides and/or other chlorine-containing compounds, it is not necessary to introduce a chlorine intermediate precursor continuously, and a discontinuous process for the introduction of the chlorine intermediate precursor can be implemented.

In aspects where the chlorine intermediate precursor is introduced continuously to the reactor, the chlorine intermediate precursor can be present in the reactant mixture in an amount of from about 0.5 vol. % to about 3 vol. %, alternatively from about 0.75 vol. % to about 3 vol. %, or alternatively from about 1 vol. % to about 3 vol. %, based on the total volume of the reactant mixture.

In aspects where the chlorine intermediate precursor is introduced continuously to the reactor, the process for producing ethylene as disclosed herein can be characterized by a reaction temperature of less than about 775° C., alternatively less than about 760° C., alternatively less than about 750° C., or alternatively about 750° C. For purposes of the disclosure herein, when the chlorine intermediate precursor is introduced continuously to the reactor, the “reaction temperature” refers to the temperature at which the reactor is operated.

In aspects where the chlorine intermediate precursor is introduced continuously to the reactor, the process for producing ethylene as disclosed herein can be characterized by a reaction temperature that is decreased when compared to a reaction temperature of an otherwise similar process conducted (i) with a reactant mixture comprising methane and oxygen and (ii) without the chlorine intermediate precursor.

In an aspect, the chlorine intermediate precursor can be introduced discontinuously to the reactor. In such aspect, the OCM catalyst can comprise the alkali metal, the alkaline earth metal, or both in an amount of equal to or greater than about 3 wt. %, alternatively from about 3 wt. % to about 20 wt. %, alternatively from about 5 wt. % to about 17.5 wt. %, alternatively from about 7.5 wt. % to about 17.5 wt. %, or alternatively from about 10 wt. % to about 15 wt. %, based on the total weight of the OCM catalyst.

In aspects where the chlorine intermediate precursor is introduced discontinuously to the reactor, the process for producing ethylene as disclosed herein can comprise (1) introducing the reactant mixture comprising the chlorine intermediate precursor to the reactor for an activation time period; (2) introducing the reactant mixture excluding the chlorine intermediate precursor to the reactor for a reaction time period; and (3) repeating steps (1) and (2) as necessary to achieve a target methane conversion and/or a target ethylene selectivity.

For purposes of the disclosure herein, when the chlorine intermediate precursor is introduced discontinuously to the reactor, the “activation time period” refers to the time period while the chlorine intermediate precursor is introduced to the reactor. In aspects where the chlorine intermediate precursor is introduced discontinuously to the reactor, the activation time period can be from about 10 minutes to about 6 hours, alternatively from about 30 minutes to about 4 hours, or alternatively from about 45 minutes to about 2 hours. As will be appreciated by one of skill in the art, and with the help of this disclosure, at the very beginning of the process for producing ethylene as disclosed herein, an activation period needs to occur, to activate the catalyst by exposing the catalyst to chlorine intermediate precursors. The activation time period is usually followed by a reaction time period.

In some aspects where the chlorine intermediate precursor is introduced discontinuously to the reactor, the chlorine intermediate precursor can be introduced to the reactor concurrently with the reactant mixture, for example as part of the reactant mixture, e.g., the chlorine intermediate precursor can be added to the reactant mixture (e.g., methane and oxygen) for the duration of the activation time period. During the activation time period, the chlorine intermediate precursor can be introduced to the reactor in an amount of from about 2 vol. % to about 5 vol. %, alternatively from about 2.5 vol. % to about 4.5 vol. %, or alternatively from about 3 vol. % to about 4 vol. %, based on the total volume of the reactant mixture. As will be appreciated by one of skill in the art, and with the help of this disclosure, in such aspects, a product mixture is still produced during the activation time period, wherein the product mixture comprises ethylene, owing to the chlorine intermediate precursor being introduced to the reactor as part of the reactant mixture.

In other aspects where the chlorine intermediate precursor is introduced discontinuously to the reactor, the flow of the reactant mixture can be stopped (e.g., discontinued) for the duration of the activation time period, and the chlorine intermediate precursor alone could be introduced to the reactor, for example in a carrier fluid such as an inert diluent (e.g., nitrogen). As will be appreciated by one of skill in the art, and with the help of this disclosure, in such aspects, a product mixture comprising ethylene is not produced during the activation time period, owing to not introducing a reactant mixture to the reactor during the activation time period.

For purposes of the disclosure herein, when the chlorine intermediate precursor is introduced discontinuously to the reactor, the “activation temperature” refers to the temperature at which the reactor is operated during the activation time period. In aspects where the chlorine intermediate precursor is introduced discontinuously to the reactor, the activation temperature can be equal to or greater than about 775° C., alternatively equal to or greater than about 800° C., alternatively equal to or greater than about 825° C., alternatively from about 800° C. to about 850° C., or alternatively equal to or greater than about 850° C.

For purposes of the disclosure herein, when the chlorine intermediate precursor is introduced discontinuously to the reactor, the “reaction time period” refers to the time period while the chlorine intermediate precursor is not introduced to the reactor. In aspects where the chlorine intermediate precursor is introduced discontinuously to the reactor, the reaction time period can be from about 1 day to about 14 days, alternatively from about 1.5 days to about 10 days, or alternatively from about 2 days to about 8 days. The reaction time period usually follows an activation time period and is followed by another activation time period, e.g., the activation time periods and the reaction time periods alternate.

For purposes of the disclosure herein, when the chlorine intermediate precursor is introduced discontinuously to the reactor, the “reaction temperature” refers to the temperature at which the reactor is operated during the reaction time period. In aspects where the chlorine intermediate precursor is introduced discontinuously to the reactor, the reaction temperature can be less than about 775° C., alternatively less than about 760° C., alternatively less than about 750° C., or alternatively about 750° C.

In aspects where the chlorine intermediate precursor is introduced discontinuously to the reactor, the process for producing ethylene as disclosed herein can be characterized by a reaction temperature that is decreased when compared to a reaction temperature of an otherwise similar process conducted (i) with a reactant mixture comprising methane and oxygen and (ii) without the chlorine intermediate precursor. In aspects where the chlorine intermediate precursor is introduced discontinuously to the reactor, a difference between the activation temperature and the reaction temperature can be equal to or greater than about 25° C., alternatively equal to or greater than about 50° C., or alternatively equal to or greater than about 75° C.

In an aspect, an amount of unreacted methane and/or ethylene in the product mixture can be periodically monitored to determine methane conversion and/or ethylene selectivity, respectively. As will be appreciated by one of skill in the art, and with the help of this disclosure, the methane conversion and the ethylene selectivity correlate with the activity of the catalyst and with the activity of the catalyst with respect to producing a desired product (e.g., ethylene), respectively. Generally, a conversion of a reagent or reactant refers to the percentage (usually mol %) of reagent that reacted to both undesired and desired products, based on the total amount (e.g., moles) of reagent present before any reaction took place. Generally, a selectivity to a desired product or products (e.g., ethylene selectivity) refers to how much desired product (e.g., ethylene) was formed divided by the total products formed, both desired and undesired (e.g., ethylene, ethane, etc.). For purposes of the disclosure herein, the selectivity to a desired product is a % selectivity based on moles converted into the desired product.

In an aspect, monitoring an amount of unreacted methane and/or ethylene in the product mixture can comprise collecting a sample of the product mixture and subjecting such sample to any suitable analytical technique for measuring and recording the amount (e.g., concentration) of unreacted methane and/or ethylene in the product mixture. Nonlimiting examples of analytical techniques suitable for measuring the amount of unreacted methane and/or ethylene in the product mixture in the present disclosure include gas chromatography (GC), mass spectrometry (MS), GC-MS, etc.

The amount of unreacted methane and/or ethylene in the product mixture can be further translated into an amount of chloride (e.g., chlorine intermediate precursor, chlorides and/or other chlorine-containing compounds, etc.) present on the catalyst (e.g., OCM catalyst), for example by using a calibration curve with known chloride amount values on the catalyst as a function of a measured amount of unreacted methane and/or ethylene in the product mixture; or as a function of the methane conversion and/or ethylene selectivity calculated from the measured amount of unreacted methane and/or ethylene, respectively, in the product mixture.

In some aspects, the end of the reaction time period can be signaled by the methane conversion and/or ethylene selectivity dropping below a predetermined threshold (e.g., methane conversion being less than about 90%, alternatively less than about 80%, or alternatively less than about 75% of a methane conversion of at the beginning of the reaction time period or at the end of the activation time period; ethylene selectivity being less than about 90%, alternatively less than about 80%, or alternatively less than about 75% of an ethylene selectivity of at the beginning of the reaction time period or at the end of the activation time period), which means that the amount of chloride present on the OCM catalyst has reached a threshold value as well.

Calibration curves can also track the time (e.g., reaction time period) it takes for the threshold amount of chloride on the catalyst to be reached, and the reaction time period can be ended at the end of a pre-determined reaction time period, even if the predetermined threshold of methane conversion and/or ethylene selectivity has not been reached, for example to maintain the process operation within known parameters. For example, the reaction time period can be ended after from about 1 day to about 14 days, alternatively after from about 1.5 days to about 10 days, or alternatively after from about 2 days to about 8 days.

Calibration curves can track the amount of chloride on the catalyst (e.g., chlorine intermediate precursor, chlorides and/or other chlorine-containing compounds, etc.) as a function of time (e.g., activation time period), and as such the activation time period can be ended when the calibration curve indicates that the time was sufficient under the activation conditions disclosed herein to reach the desired amount of chloride on the OCM catalyst (e.g., chlorinated OCM catalyst). For example, the activation time period can be ended after from about 10 minutes to about 6 hours, alternatively after from about 30 minutes to about 4 hours, or alternatively after from about 45 minutes to about 2 hours. The desired amount of chloride on the OCM catalyst can be generally based on the desired catalyst activity, which in turn can be assessed with a calibration curve of known chloride amount values on the catalyst as a function of a measured amount of unreacted methane and/or ethylene in the product mixture; or as a function of the methane conversion and/or ethylene selectivity calculated from the measured amount of unreacted methane and/or ethylene in the product mixture, respectively.

In an aspect, a process for producing ethylene as disclosed herein can comprise recovering at least a portion of the product mixture from the reactor, wherein the product mixture can be collected as an outlet gas mixture from the reactor.

In an aspect, the product mixture can be characterized by an ethylene to ethane molar ratio that is increased when compared to an ethylene to ethane molar ratio of a product mixture produced via an otherwise similar process conducted (i) with a reactant mixture comprising methane and oxygen and (ii) without the chlorine intermediate precursor.

In an aspect, the product mixture can be characterized by an ethylene to ethane molar ratio of equal to or greater than about 6:1, alternatively equal to or greater than about 8:1, alternatively equal to or greater than about 8:1, or alternatively from about 6:1 to about 8:1.

In an aspect, the product mixture can comprises less than about 15 mol %, alternatively less than about 12.5 mol %, or alternatively less than about 10 mol % carbon dioxide (CO₂). The process for producing ethylene as disclosed herein can comprise minimizing deep oxidation of methane to CO₂.

In an aspect, the product mixture can be characterized by a carbon monoxide to carbon dioxide molar ratio that is increased when compared to a carbon monoxide to carbon dioxide molar ratio of a product mixture produced via an otherwise similar process conducted (i) with a reactant mixture comprising methane and oxygen and (ii) without the chlorine intermediate precursor.

In an aspect, the product mixture can be characterized by a carbon monoxide to carbon dioxide molar ratio of equal to or greater than about 0.8:1, alternatively equal to or greater than about 1.5:1, alternatively equal to or greater than about 3:1, or alternatively equal to or greater than about 5:1.

In an aspect, a process for producing ethylene as disclosed herein can comprise recovering at least a portion of C₂ hydrocarbons from the product mixture. The product mixture can comprise C₂₊ hydrocarbons (including olefins), unreacted methane, and optionally a diluent. The water produced from the OCM reaction and the water used as a diluent (if water diluent is used) can be separated from the product mixture prior to separating any of the other product mixture components. For example, by cooling down the product mixture to a temperature where the water condenses (e.g., below 100° C. at ambient pressure), the water can be removed from the product mixture, by using a flash chamber for example.

In an aspect, at least a portion of the C₂₊ hydrocarbons can be separated (e.g., recovered) from the product mixture to yield recovered C₂₊ hydrocarbons. The C₂₊ hydrocarbons can be separated from the product mixture by using any suitable separation technique. In an aspect, at least a portion of the C₂₊ hydrocarbons can be separated from the product mixture by distillation (e.g., cryogenic distillation).

In an aspect, at least a portion of the recovered C₂₊ hydrocarbons can be used for ethylene production. In some aspects, at least a portion of ethylene can be separated from the product mixture (e.g., from the C₂₊ hydrocarbons, from the recovered C₂₊ hydrocarbons) to yield recovered ethylene and recovered hydrocarbons, by using any suitable separation technique (e.g., distillation). In other aspects, at least a portion of the recovered hydrocarbons (e.g., recovered C₂+ hydrocarbons after olefin separation, such as separation of C₂H₄ and C₃H₆) can be converted to ethylene, for example via a conventional steam cracking process.

In an aspect, at least a portion of the unreacted methane can be separated from the product mixture to yield recovered methane. Methane can be separated from the product mixture by using any suitable separation technique, such as for example distillation (e.g., cryogenic distillation). At least a portion of the recovered methane can be recycled to the reactant mixture.

In some aspects, at least a portion of the carbon monoxide can be separated from the product mixture to yield recovered carbon monoxide. The recovered carbon monoxide can be used in syngas, and the syngas can be further used for a variety of processes, such as methanol production processes.

In an aspect, a process for producing ethylene can comprise (a) continuously feeding a reactant mixture to a reactor to yield a product mixture, wherein the reactor comprises an OCM catalyst, wherein the reactor is characterized by a reaction temperature of about 750° C., wherein the reactant mixture comprises methane, oxygen, and HCl, wherein the HCl is present in the reactant mixture in an amount of from about 0.5 vol. % to about 3 vol. %, based on the total volume of the reactant mixture, wherein the product mixture comprises ethylene, ethane, and unreacted methane, wherein the OCM catalyst comprises (1) manganese (Mn) in an amount of from about 10 wt. % to about 20 wt. %, based on the total weight of the OCM catalyst, (2) sodium (Na), calcium (Ca), or both in an amount of from about 1 wt. % to about 3 wt. %, based on the total weight of the OCM catalyst, and (3) a SiO₂ support; and (b) recovering at least a portion of the ethylene from the product mixture. In such aspect, the product mixture can be characterized by an ethylene to ethane molar ratio of equal to or greater than about 6:1.

In an aspect, a process for producing ethylene can comprise (a) continuously feeding a reactant mixture and HCl for an activation time period of from about 45 minutes to about 2 hours to a reactor comprising an OCM catalyst to activate the OCM catalyst and to yield a first product mixture, wherein the reactant mixture comprises methane and oxygen, wherein the first product mixture comprises ethylene, ethane, and unreacted methane, wherein the OCM catalyst comprises (1) manganese (Mn) in an amount of from about 15 wt. % to about 25 wt. %, based on the total weight of the OCM catalyst, (2) sodium (Na), calcium (Ca), or both in an amount of from about 10 wt. % to about 20 wt. %, based on the total weight of the OCM catalyst, and (3) a SiO₂ support; (b) discontinuing the introduction of HCl to the reactor while continuing to feed the reactant mixture to the reactor for a reaction time period of from about 2 days to about 8 days to produce a second product mixture, wherein the second product mixture comprises ethylene, ethane, and unreacted methane; (c) repeating steps (a) and (b) as necessary to achieve a target methane conversion and/or a target ethylene selectivity; and (d) recovering at least a portion of the ethylene from the first product mixture and/or the second product mixture. In such aspect, step (a) can occur at an activation temperature of from about 800° C. to about 850° C.; and step (b) can occur at a reaction temperature of less than about 750° C. The HCl can be introduced to the reactor in step (a) in an amount of from about 2 vol. % to about 5 vol. %, based on the total volume of the reactant mixture. The first product mixture and/or the second product mixture can be characterized by an ethylene to ethane molar ratio that is increased when compared to an ethylene to ethane molar ratio of a product mixture produced via an otherwise similar process conducted with an OCM catalyst that has not been activated via a chlorine radical precursor (e.g., HCl). In some aspects, the first product mixture and/or the second product mixture can be characterized by an ethylene to ethane molar ratio of equal to or greater than about 8:1.

In an aspect, a process for producing ethylene as disclosed herein can advantageously display improvements in one or more process characteristics when compared to an otherwise similar process conducted with an OCM catalyst that has not been activated via a chlorine intermediate precursor. The process for producing ethylene as disclosed herein can advantageously minimize deep oxidation reactions to CO₂. In an aspect, a process for producing ethylene as disclosed herein can advantageously be characterized by an amount of carbon dioxide in the product mixture that is decreased when compared to an amount of carbon dioxide in a product mixture produced via an otherwise similar process conducted (i) with a reactant mixture comprising methane and oxygen and (ii) without the chlorine intermediate precursor.

In an aspect, a process for producing ethylene as disclosed herein can advantageously be characterized by a methane conversion that is increased when compared to a methane conversion of an otherwise similar process conducted (i) with a reactant mixture comprising methane and oxygen and (ii) without the chlorine intermediate precursor.

In an aspect, a process for producing ethylene as disclosed herein can advantageously be characterized by an ethylene selectivity that is increased when compared to an ethylene selectivity of an otherwise similar process conducted (i) with a reactant mixture comprising methane and oxygen and (ii) without the chlorine intermediate precursor.

In an aspect, a process for producing ethylene as disclosed herein can advantageously be characterized by an amount of ethane in the product mixture that is decreased when compared to an amount of ethane in a product mixture produced via an otherwise similar process conducted (i) with a reactant mixture comprising methane and oxygen and (ii) without the chlorine intermediate precursor.

In aspects where the chlorine intermediate precursor is introduced discontinuously to the reactor, a process for producing ethylene as disclosed herein can advantageously reduce corrosion to the reactor, owing to shorter contact times of the chlorine intermediate precursor with inner surfaces of the reactor.

In an aspect, a process for producing ethylene as disclosed herein can advantageously be characterized by an overall higher efficiency (e.g., for example in terms of cost) when compared to a two-step process of methane conversion to olefins via (1) oxychlorination of methane to methyl chloride, followed by (2) conversion of methyl chloride to ethylene. Further, the oxychlorination uses more HCl because it needs to convert the methane to methyl chloride, while the process for producing ethylene as disclosed herein can use a very small amount of HCl (comparatively), as the HCl does not participate in the overall reactions, it functions as a promoter that is not incorporated into the final products, as represented in equations (10)-(15). Additional advantages of the processes for the production of ethylene as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

An OCM catalyst was prepared as follows. 10 g of dried silica-gel (drying procedure: 120° C., 2 hours) was impregnated with the necessary amount of Mn(NO₃)₂.4H₂O and the necessary amount of Na₂CO₃. 2H₂O to get a catalyst composition comprising x % Na-y % Mn/SiO₂. A solution of of Mn(NO₃)₂.4H₂O and Na₂CO₃.2H₂O in water was added to 20 g SiO₂ and heated at 45° C. with continuous stirring. After impregnation, the resulting solid catalyst material was oven dried in air atmosphere for 12 hours at 120 ° C., and then was calcined for 4 hours in air atmosphere at 850° C. The dried and calcined catalyst was crushed to produce an OCM catalyst with a particle size of 20-50 mesh, and was used in the following examples.

### Example 2

Oxidative conversion reactions of methane to a mixture of C₂ hydrocarbons were conducted in the presence of a catalyst as follows. The reactant mixture comprised methane and air. The used catalyst was 2% Na-15% Mn/SiO₂, prepared by using the procedure described in Example 1, with 13.6 g Mn(NO₃)₂.4H₂O and 1.23 g Na₂CO₃.2H₂O, supported on 20 g of SiO₂; and the reaction temperature was 750° C. The reactor was a quartz fixed bed reactor with a diameter of 7 mm and a length of 12 cm heated by electrical heating, with a catalyst loading of 3 ml. The reactions were conducted at CH₄/O₂=2, 1 mol % HCl in the reactant mixture, and at a gas hourly space velocity (GHSV) of 7,200 h⁻¹.

The CH₄ conversion was 43.5%. For example, the methane conversion can be calculated by using the following equation:

$${CH}_{4} conversion = \frac{{C}_{{CH}_{4}}^{in} - {C}_{{CH}_{4}}^{out}}{{C}_{{CH}_{4}}^{in}} \times 100 \%$$

wherein C_{CH}_{₄}ⁱⁿ=number of moles of C from CH₄ that entered the reactor as part of the reactant mixture; and C_{CH}_{₄}^{out}=number of moles of C from CH₄ that was recovered from the reactor as part of the product mixture.

The C₂H₄ selectivity was 55.0%; the C₂H₆ selectivity was 3.6%; the CO selectivity was 22.0%; and the CO₂ selectivity was 19.4%. As it can be seen from the data in Example 2, the C₂ hydrocarbons comprise mostly ethylene.

### Example 3

Oxidative conversion reactions of methane to a mixture of C₂ hydrocarbons were conducted in the presence of a catalyst as follows. The reactant mixture comprised methane and air. The used catalyst was 8% Na-15% Mn/SiO₂, prepared by using the procedure described in Example 1, with 12 g Mn(NO₃)₂.4H₂O and 4.92 g Na₂CO₃.2H₂O supported on 20 g of SiO₂; and the reaction temperature was 750° C. The reactor was a quartz fixed bed reactor with a diameter of 7 mm and a length of 12 cm heated by electrical heating, with a catalyst loading of 3 ml. The reactions were conducted at CH₄/O₂=2, 1 mol % HCl in the reactant mixture, and at a GHSV of 7,200 h⁻¹.

The CH₄ conversion was 22.0%. The C₂H₄ selectivity was 32.3%; the C₂H₆ selectivity was 4.0%; the CO selectivity was 51.5%; and the CO₂ selectivity was 12.2%. Increasing the amount of Na in the catalyst led to a decrease in methane conversion as compared to Example 2, along with an increase in CO selectivity.

### Example 4

Oxidative conversion reactions of methane to a mixture of C₂ hydrocarbons were conducted in the presence of a catalyst as follows. The reactant mixture comprised methane and air. The used catalyst was 15% Na-15% Mn/SiO₂, prepared by using the procedure described in Example 1, with 12 g Mn(NO₃)₂.4H₂O and 8.8 g Na₂CO₃.2H₂O supported on 20 g of SiO₂; and subsequently treated for 1 hour with CH4+air+3 mol % HCl at 850° C. The reactor was a quartz fixed bed reactor with a diameter of 7 mm and a length of 12 cm heated by electrical heating, with a catalyst loading of 3 ml. After the catalyst was activated with HCl, the gas mixture containing CH4+air+3 mol % HCl was replaced with CH4+air, and the temperature was reduced to 750° C. The reactions were conducted at CH₄/O₂=2, and at a GHSV of 7,200 h⁻¹.

The CH₄ conversion was 41.5%. The C₂H₄ selectivity was 60.5%; the C₂H₆ selectivity was 6.2%; the CO selectivity was 19.7%; and the CO₂ selectivity was 13.6%. Further increasing the amount of Na in the catalyst and treating the catalyst discontinuously with HCl led to an increase in methane conversion as compared to Example 3, almost to the level of Example 2, while producing an increase in C₂H₄ selectivity.

### Example 5

Oxidative conversion reactions of methane to a mixture of C₂ hydrocarbons were conducted in the presence of a catalyst as follows. The reactant mixture comprised methane and air. The used catalyst was 2% Na-15% Mn/SiO₂, prepared by using the procedure described in Example 1, with 12 g Mn(NO₃)₂4H₂O and 1.23 g Na₂CO₃2H₂O supported on 20 g of SiO₂; and the reaction temperature was 850° C. The reactor was a quartz fixed bed reactor with a diameter of 7 mm and a length of 12 cm heated by electrical heating, with a catalyst loading of 3 ml. The reactions were conducted at CH₄/O₂=2, 1 mol % HCl in the reactant mixture, and at a GHSV of 7,200 h⁻¹.

The CH₄ conversion was 30.5%. The C₂H₄ selectivity was 23.0%; the C₂H₆ selectivity was 12.5%; the CO selectivity was 5.2%; and the CO₂ selectivity was 59.3%. Increasing the reaction temperature (as compared to Example 2) led to a decrease in methane conversion, along with a drastic increase in C₂H₆ selectivity and CO₂ selectivity.

### Example 6

Catalysts for oxidative conversion reactions of methane to a mixture of C₂ hydrocarbons could be a mixture of alkali metals and/or alkaline earth metal, and redox elements such as Na—Mn—O/SiO₂ and/or Ca—Mn—O/SiO₂ in the form of catalyst supported on SiO₂. Concentrations of Na or Ca in the catalysts Na—Mn—O/SiO₂ and Ca—MnO/SiO₂ could vary from 3 wt. % to 15 wt. %, and concentrations of Mn in the catalysts Na—Mn—O/SiO₂ and Ca—MnO/SiO₂ could vary from 3 wt. % to 20 wt. %, based on the total weight of the catalyst.

At low concentrations of alkali metals and/or alkaline earth metals, such as 1-2% Na-(15-20%)Mn—O/SiO₂ and/or 1-2% Ca-(15-20%)Mn—O/SiO₂, addition of chlorine containing components (e.g., chlorine intermediate precursors) to the reactant mixture would lead to an increase in methane conversion, C₂ selectivity and C₂H₄/C₂H₆ molar ratio. At high concentrations of alkali metals and/or alkaline earth metals, addition of chlorine containing components to the reactant mixture would lead to a decrease in methane conversion. However, at high concentrations of alkali metals and/or alkaline earth metals, stopping addition of chlorine containing components to the reactant mixture would lead to an increase in methane conversion, C₂ selectivity and C₂H₄/C₂H₆ molar ratio. Without wishing to be limited by theory, the increase in methane conversion, C₂ selectivity and C₂H₄/C₂H₆ molar ratio upon stopping addition of chlorine containing components to the reactant mixture could be explained by blockage of the catalyst surface with NaCl or CaCl₂, which could form at high alkali metal and/or alkaline earth metal concentrations. Further, and without wishing to be limited by theory, stopping addition of chlorine containing components to the reactant mixture can lead to migration of chlorine from the catalyst surface into the gas phase, which in turn leads to an increase in methane conversion through gas phase radical reactions. 4-5 days after stopping addition of chlorine containing components to the reactant mixture, catalyst activity would decrease, and C₂ selectivity would decrease to the value which was observed for oxide form of a catalyst without addition of chlorine containing components to the reactant mixture. Reintroducing chlorine containing components to the reactant mixture for about 1 hour would restore high activity for the catalyst, wherein after stopping again the addition of chlorine containing components to the reactant mixture, increased conversion and increased selectivity would be observed. The process of restoring catalyst activity via introducing chlorine containing components to the reactant mixture would be performed when a decrease in conversion and selectivity due to the removal of chlorine from the catalyst would be observed. Such decrease in conversion and selectivity would be gradual and would take place over several days, before it would become necessary to re-activate the catalyst by exposing it to chlorine containing components.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### ADDITIONAL DISCLOSURE

A first aspect, which is a process for producing ethylene comprising (a) contacting a reactant mixture with an oxidative coupling of methane (OCM) catalyst in the presence of a chlorine intermediate precursor in a reactor to yield a product mixture, wherein the reactant mixture comprises methane and oxygen, wherein the product mixture comprises ethylene, ethane, and unreacted methane, and wherein the OCM catalyst comprises an alkali metal, an alkaline earth metal, or both; and (b) recovering at least a portion of the ethylene from the product mixture.

A second aspect, which is the process of the first aspect, wherein yielding the product mixture in step (a) further comprises (i) allowing a first portion of the reactant mixture to react via an OCM reaction, (ii) allowing at least a portion of the chlorine intermediate precursor to generate a chlorine intermediate, and (iii) allowing a second portion of the reactant mixture to react via the chlorine intermediate.

A third aspect, which is the process of the second aspect, wherein step (ii) further comprises (ii)(1) contacting at least a portion of the chlorine intermediate precursor with the OCM catalyst to form a chlorinated OCM catalyst; and (ii)(2) allowing at least a portion of the chlorinated OCM catalyst to generate the chlorine intermediate.

A fourth aspect, which is the process of any one of the first through the third aspects, wherein the chlorine intermediate precursor is a chlorine radical precursor, and wherein chlorine intermediate is a chlorine radical.

A fifth aspect, which is the process of any one of the first through the fourth aspects, wherein the chlorine intermediate precursor is introduced continuously to the reactor.

A sixth aspect, which is the process of the fifth aspect, wherein the OCM catalyst comprises the alkali metal, the alkaline earth metal, or both in an amount of less than about 3 wt. %, based on the total weight of the OCM catalyst.

A seventh aspect, which is the process of any one of the first through the fourth aspects, wherein the chlorine intermediate precursor is introduced discontinuously to the reactor.

An eighth aspect, which is the process of the seventh aspect, wherein the OCM catalyst comprises the alkali metal, the alkaline earth metal, or both in an amount of equal to or greater than about 3 wt. %, based on the total weight of the OCM catalyst.

A ninth aspect, which is the process of any one of the seventh and the eighth aspects, wherein the OCM catalyst comprises the alkali metal, the alkaline earth metal, or both in an amount of from about 3 wt. % to about 20 wt. %, based on the total weight of the OCM catalyst.

A tenth aspect, which is the process of any one of the seventh through the ninth aspects, wherein step (a) further comprises (1) introducing the reactant mixture comprising the chlorine intermediate precursor to the reactor for an activation time period; (2) introducing the reactant mixture excluding the chlorine intermediate precursor to the reactor for a reaction time period; and (3) repeating steps (1) and (2) as necessary to achieve a target methane conversion and/or a target ethylene selectivity.

An eleventh aspect, which is the process of the tenth aspect, wherein the activation time period is from about 10 minutes to about 6 hours.

A twelfth aspect, which is the process of any one of the seventh through the eleventh aspects, wherein the reaction time period is from about 1 day to about 14 days.

A thirteenth aspect, which is the process of any one of the seventh through the twelfth aspects, wherein the process is characterized by an activation temperature during step (1) and by a reaction temperature during step (2), and wherein the activation temperature is greater than the reaction temperature.

A fourteenth aspect, which is the process of the thirteenth aspect, wherein a difference between the activation temperature and the reaction temperature is equal to or greater than about 25° C.

A fifteenth aspect, which is the process of any one of the seventh through the fourteenth aspects, wherein the activation temperature is equal to or greater than about 775° C.

A sixteenth aspect, which is the process of any one of the seventh through the fifteenth aspects, wherein the reaction temperature is less than about 775° C.

A seventeenth aspect, which is the process of any one of the first through the sixteenth aspects, wherein the chlorine intermediate precursor is introduced to the reactor in an amount of from about 0.5 vol. % to about 5 vol. %, based on the total volume of the reactant mixture.

An eighteenth aspect, which is the process of any one of the first through the seventeenth aspects, wherein the chlorine intermediate precursor comprises hydrogen chloride, methyl chloride, methylene chloride, chloroform, carbon tetrachloride, ethyl chloride, 1,2-dichloroethane, trichloroethylene, or combinations thereof.

A nineteenth aspect, which is the process of any one of the first through the eighteenth aspects, wherein the alkali metal comprises sodium (Na), potassium (K), rubidium (Rb), cesium (Cs), or combinations thereof.

A twentieth aspect, which is the process of any one of the first through the nineteenth aspects, wherein the alkaline earth metal comprises magnesium (Mg), calcium (Ca), strontium (Sr), barium (Ba), or combinations thereof.

A twenty-first aspect, which is the process of any one of the first through the twentieth aspects, wherein the alkali metal is sodium (Na).

A twenty-second aspect, which is the process of any one of the first through the twenty-first aspects, wherein the alkaline earth metal is calcium (Ca).

A twenty-third aspect, which is the process of any one of the first through the twenty-second aspects, wherein the OCM catalyst further comprises a redox agent.

A twenty-fourth aspect, which is the process of the twenty-third aspect, wherein the redox agent comprises manganese (Mn), tin (Sn), bismuth (Bi), cerium (Ce), or combinations thereof.

A twenty-fifth aspect, which is the process of any one of the first through the twenty-fourth aspects, wherein the redox agent is manganese (Mn).

A twenty-sixth aspect, which is the process of any one of the first through the twenty-fifth aspects, wherein the redox agent is present in the OCM catalyst in an amount of from about 1 wt. % to about 25 wt. %, based on the total weight of the OCM catalyst.

A twenty-seventh aspect, which is the process of any one of the first through the twenty-sixth aspects, wherein the process is characterized by a reaction temperature that is decreased when compared to a reaction temperature of an otherwise similar process conducted (i) with a reactant mixture comprising methane and oxygen and (ii) without the chlorine intermediate precursor.

A twenty-eighth aspect, which is the process of any one of the first through the twenty-seventh aspects, wherein the process is characterized by a reaction temperature of less than about 750° C.

A twenty-ninth aspect, which is the process of any one of the first through the twenty-eighth aspects, wherein the product mixture is characterized by an ethylene to ethane molar ratio that is increased when compared to an ethylene to ethane molar ratio of a product mixture produced via an otherwise similar process conducted (i) with a reactant mixture comprising methane and oxygen and (ii) without the chlorine intermediate precursor.

A thirtieth aspect, which is the process of any one of the first through the twenty-ninth aspects, wherein the product mixture is characterized by an ethylene to ethane molar ratio of equal to or greater than about 6:1.

A thirty-first aspect, which is the process of any one of the first through the thirtieth aspects, wherein the OCM catalyst comprises one or more oxides.

A thirty-second aspect, which is the process of the thirty-first aspect, wherein the one or more oxides comprises oxides of rare earth elements.

A thirty-third aspect, which is the process of any one of the first through the thirty-second aspects, wherein the OCM catalyst comprises CeO₂, La₂O₃—CeO₂, Ca/CeO₂, Mn/Na₂WO₄, Li₂O, Na₂O, Cs₂O, WO₃, Mn₃O₄, CaO, MgO, SrO, BaO, CaO—MgO, CaO—BaO, Li/MgO, MnO, Ca—Mn—O/SiO₂, W₂O₃, SnO₂, Yb₂O₃, Sm₂O₃, MnO—W₂O₃, MnO—W₂O₃—Na₂O, MnO—W₂O₃—Li₂O, SrO/La₂O₃, La₂O₃, Ce₂O₃, La/MgO, La₂O₃—CeO₂—Na₂O, La₂O₃—CeO₂—CaO, Na₂O—MnO—WO₃—La₂O₃, La₂O₃—CeO₂—MnO—WO₃—SrO, Na—Mn—La₂O₃/Al₂O₃, Na—Mn/SiO₂, Na—Mn—O/SiO₂, Na₂WO₄—Mn/SiO₂, Na₂WO₄—Mn—O/SiO₂, Na/Mn/O, Na₂WO₄, Mn₂O₃/Na₂WO₄, Mn₃O₄/Na₂WO₄, MnWO₄/Na₂WO₄, MnWO₄/Na₂WO₄ Mn/WO₄, Na₂WO₄/Mn, Sr/Mn—Na₂WO₄, or combinations thereof.

A thirty-fourth aspect, which is the process of any one of the first through the thirty-third aspects, wherein the OCM catalyst further comprises a support.

A thirty-fifth aspect, which is the process of the thirty-fourth aspect, wherein at least a portion of the OCM catalyst contacts, coats, is embedded in, is supported by, and/or is distributed throughout at least a portion of the support; and wherein the support comprises MgO, Al₂O₃, SiO₂, ZrO₂, or combinations thereof.

A thirty-sixth aspect, which is the process of any one of the first through the thirty-fifth aspects further comprising minimizing deep oxidation of methane to carbon dioxide (CO₂).

A thirty-seventh aspect, which is the process of any one of the first through the thirty-sixth aspects, wherein the product mixture comprises less than about 15 mol % carbon dioxide (CO₂).

A thirty-eighth aspect, which is the process of any one of the first through the thirty-seventh aspect, wherein the product mixture is characterized by a carbon monoxide to carbon dioxide molar ratio that is increased when compared to a carbon monoxide to carbon dioxide molar ratio of a product mixture produced via an otherwise similar process conducted (i) with a reactant mixture comprising methane and oxygen and (ii) without the chlorine intermediate precursor.

A thirty-ninth aspect, which is the process of any one of the first through the thirty-eighth aspect, wherein the process is characterized by a methane conversion that is increased when compared to a methane conversion of an otherwise similar process conducted (i) with a reactant mixture comprising methane and oxygen and (ii) without the chlorine intermediate precursor.

A fortieth aspect, which is the process of any one of the first through the thirty-ninth aspect, wherein the process is characterized by an ethylene selectivity that is increased when compared to an ethylene selectivity of an otherwise similar process conducted (i) with a reactant mixture comprising methane and oxygen and (ii) without the chlorine intermediate precursor.

A forty-first aspect, which is a process for producing ethylene comprising (a) continuously feeding a reactant mixture to a reactor to yield a product mixture, wherein the reactor comprises an oxidative coupling of methane (OCM) catalyst, wherein the reactant mixture comprises methane, oxygen, and a chlorine radical precursor, wherein the product mixture comprises ethylene, ethane, and unreacted methane, wherein the OCM catalyst comprises (1) a redox agent in an amount of from about 1 wt. % to about 25 wt. %, based on the total weight of the OCM catalyst, and (2) an alkali metal, an alkaline earth metal, or both, in an amount of less than about 3 wt. %, based on the total weight of the OCM catalyst; and (b) recovering at least a portion of the ethylene from the product mixture.

A forty-second aspect, which is the process of the forty-first aspect, wherein the chlorine radical precursor is present in the reactant mixture in an amount of from about 0.5 vol. % to about 3 vol. %, based on the total volume of the reactant mixture.

A forty-third aspect, which is the process of any one of the forty-first and the forty-second aspects, wherein yielding the product mixture in step (a) further comprises (i) allowing a first portion of the methane of the reactant mixture to react via an OCM reaction, (ii) allowing at least a portion of the chlorine radical precursor to generate a chlorine radical, and (iii) allowing a second portion of the methane of the reactant mixture to react via the chlorine radical.

A forty-fourth aspect, which is the process of any one of the forty-first through the forty-third aspects, wherein the process is characterized by a reaction temperature that is decreased when compared to a reaction temperature of an otherwise similar process conducted with a reactant mixture comprising methane and oxygen without the chlorine radical precursor.

A forty-fifth aspect, which is the process of any one of the forty-first through the forty-fourth aspects, wherein the process is characterized by a reaction temperature of less than about 750° C.

A forty-sixth aspect, which is the process of any one of the forty-first through the forty-fifth aspects, wherein the product mixture is characterized by an ethylene to ethane molar ratio that is increased when compared to an ethylene to ethane molar ratio of a product mixture produced via an otherwise similar process conducted with a reactant mixture comprising methane and oxygen without the chlorine radical precursor.

A forty-seventh aspect, which is the process of any one of the forty-first through the forty-sixth aspects, wherein the product mixture is characterized by an ethylene to ethane molar ratio of equal to or greater than about 6:1.

A forty-eighth aspect, which is the process of any one of the forty-first through the forty-seventh aspects, wherein the chlorine radical precursor comprises hydrogen chloride (HCl); and wherein the OCM catalyst comprises (1) manganese (Mn) in an amount of from about 10 wt. % to about 20 wt. %, based on the total weight of the OCM catalyst, (2) sodium (Na), calcium (Ca), or both in an amount of from about 1 wt. % to about 3 wt. %, based on the total weight of the OCM catalyst, and (3) a SiO₂ support.

A forty-ninth aspect, which is a process for producing ethylene comprising (a) continuously feeding a reactant mixture and a chlorine radical precursor for an activation time period to a reactor comprising an oxidative coupling of methane (OCM) catalyst to activate the OCM catalyst and to yield a first product mixture, wherein the reactant mixture comprises methane and oxygen, wherein the first product mixture comprises ethylene, ethane, and unreacted methane, wherein the OCM catalyst comprises (1) a redox agent in an amount of from about 1 wt. % to about 25 wt. %, based on the total weight of the OCM catalyst, and (2) an alkali metal, an alkaline earth metal, or both, in an amount of equal to or greater than about 3 wt. %, based on the total weight of the OCM catalyst; (b) discontinuing the introduction of the chlorine radical precursor to the reactor while continuing to feed the reactant mixture to the reactor for a reaction time period to produce a second product mixture, wherein the second product mixture comprises ethylene, ethane, and unreacted methane; (c) repeating steps (a) and (b) as necessary to achieve a target methane conversion and/or a target ethylene selectivity; and (d) recovering at least a portion of the ethylene from the first product mixture and/or the second product mixture.

A fiftieth aspect, which is the process of the forty-ninth aspect, wherein the activation time period is from about 10 minutes to about 6 hours.

A fifty-first aspect, which is the process of anyone of the forty-ninth and the fiftieth aspect, wherein the reaction time period is from about 1 day to about 14 days.

A fifty-second aspect, which is the process of anyone of the forty-ninth through the fifty-first aspects, wherein step (a) occurs at an activation temperature, and wherein step (b) occurs at a reaction temperature, and wherein the activation temperature is greater than the reaction temperature.

A fifty-third aspect, which is the process of the fifty-second aspect, wherein a difference between the activation temperature and the reaction temperature is equal to or greater than about 50° C.

A fifty-fourth aspect, which is the process of anyone of the forty-ninth through the fifty-third aspects, wherein the activation temperature is equal to or greater than about 800° C.

A fifty-fifth aspect, which is the process of anyone of the forty-ninth through the fifty-fourth aspects, wherein the activation temperature is from about 800° C. to about 850° C.

A fifty-sixth aspect, which is the process of anyone of the forty-ninth through the fifty-fifth aspects, wherein the reaction temperature is less than about 750° C.

A fifty-seventh aspect, which is the process of anyone of the forty-ninth through the fifty-sixth aspects, wherein the process is characterized by a reaction temperature that is decreased when compared to a reaction temperature of an otherwise similar process conducted with an OCM catalyst that has not been activated via the chlorine radical precursor.

A fifty-eighth aspect, which is the process of anyone of the forty-ninth through the fifty-seventh aspects, wherein during step (a) the chlorine radical precursor is introduced to the reactor in an amount of from about 2 vol. % to about 5 vol. %, based on the total volume of the reactant mixture

A fifty-ninth aspect, which is the process of anyone of the forty-ninth through the fifty-eighth aspects, wherein the first product mixture and/or the second product mixture are characterized by an ethylene to ethane molar ratio that is increased when compared to an ethylene to ethane molar ratio of a product mixture produced via an otherwise similar process conducted with an OCM catalyst that has not been activated via the chlorine radical precursor.

A sixtieth aspect, which is the process of anyone of the forty-ninth through the fifty-ninth aspects, wherein the first product mixture and/or the second product mixture are characterized by an ethylene to ethane molar ratio of equal to or greater than about 8:1.

A sixty-first aspect, which is the process of anyone of the forty-ninth through the sixtieth aspects, wherein the chlorine radical precursor comprises hydrogen chloride (HCl); and wherein the OCM catalyst comprises (1) manganese (Mn) in an amount of from about 15 wt. % to about 25 wt. %, based on the total weight of the OCM catalyst, (2) sodium (Na), calcium (Ca), or both in an amount of from about 10 wt. % to about 20 wt. %, based on the total weight of the OCM catalyst, and (3) a SiO₂ support.

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

1. A process for producing ethylene comprising:
(a) contacting a reactant mixture with an oxidative coupling of methane (OCM) catalyst in the presence of a chlorine intermediate precursor in a reactor to yield a product mixture, wherein the reactant mixture comprises methane and oxygen, wherein the product mixture comprises ethylene, ethane, and unreacied methane, and wherein the OCM catalyst comprises an alkali metal, an alkaline earth metal, or both; and
(b) recovering at least a portion of the ethylene from the product mixture.

2. The process of claim 1, wherein yielding the product mixture in step (a) further comprises (i) allowing a first portion of the reactant mixture to react via an OCM reaction, (ii) allowing at least a portion of the chlorine intermediate precursor to generate a chlorine intermediate, and (iii) allowing a second portion of the reactant mixture to react via the chlorine intermediate.

3. The process of claim 2, wherein step (ii) further comprises (ii)(1) contacting at least a portion of the chlorine intermediate precursor with the OCM catalyst to form a chlorinated OCM catalyst; and (ii)(2) allowing at least a portion of the chlorinated OCM catalyst to generate the chlorine intermediate.

4. The process of claim 1, wherein the chlorine intermediate precursor is a chlorine radical precursor, and wherein chlorine intermediate is a chlorine radical.

5. The process of claim 1, wherein the chlorine intermediate precursor is introduced continuously to the reactor; and wherein the OCM catalyst comprises the alkali metal, the alkaline earth metal or both in an amount of less than about 3 wt. %, based on the total weight of the OCM catalyst.

6. The process of claim 1, wherein the chlorine intermediate precursor is introduced discontinuously to the reactor, and wherein the OCM catalyst comprises the alkali mctai, the alkaline earth metal, or both in an amount of equal to or greater than about 3 wt. %, based on the total weight of the OCM catalyst.

7. The process of claim 6, wherein step (a) further comprises (1) introducing the reactant mixture comprising the chlorine intermediate precursor to the reactor for an activation time period; (2) introducing the reactant mixture excluding the chlorine intermediate precursor to the reactor for a reaction time period; and (3) repeating steps (1) and (2) as necessary to achieve a target methane conversion and/or a target ethylene selectivity.

8. The process of claim 7, wherein the activation time period is from about 10 minutes to about 6 hours; and wherein the reaction time period is from about 1 day to about 14 days.

9. The process of claim 6, wherein the process is characterized by an activation temperature during step (1) and by a reaction temperature during step (2); wherein the activation temperature is greater than the reaction temperature; and wherein a difference between the activation temperature and the reaction temperature is equal to or greater than about 25° C.

10. The process of claim 6, wherein the activation temperature is equal to or greater than about 775° C.; and wherein the reaction temperature is less than about 775° C.

11. The process of claim 1, wherein the chlorine intermediate precursor is introduced to the reactor in an amount of from about 0.5 vol. % to about 5 vol. %, based on the total volume of tltc reactant mixture; and wherein the chlorine intermediate precursor comprises hydrogen chloride, methyl chloride, methylene chloride, chloroform, carbon tetrachloride, ethyl chloride, 1,2-dichloroethane, trichloroetltylene, or combinations thereof.

12. The process of claim 1, wherein the alkali metal comprises sodium (Na), potassiiun (K), rubidium (Kb), cesium (Cs), or combinations thereof; and wherein the alkaline earth metal comprises magnesium (Mg), calcium (Ca), strontium (Sr), barium (Ba), or combinations thereof.

13. The process of claim 1, wherein the OCM catalyst further comprises a redox agent; wherein the redox agent is present in the OCM catalyst in an amount of from about 1 wt. % to about 25 wt. %, based on the total weight of the OCM catalyst; and wherein the redox agent comprises manganese (Mn), tin (Sn), bismuth (Bi), cerium (Ce), or combinations thereof.

14. The process of claim 1, wherein the product mixture is characterized by an ethylene to ethane molar ratio of equal to or greater than about 6:1.

15. The process of claim 1, wherein the OCM catalyst comprises one or more oxides.

16. The process of claim 1, wherein the process is characterized by a methane conversion that is increased when compared to a methane conversion of an otherwise similar process conducted (i) with a reactant mixture comprising methane and oxygen and (ii) without the chlorine intermediate precursor; and wherein the process is characterized by an ethylene selectivity that is increased when compared to an ethylene selectivity of an otherwise similar process conducted (i) with a reactant mixture comprising methane and oxygen and (ii) without the chlorine intermediate precursor.

17. A process for producing ethylene comprising:
(a) continuously feeding a reactant mixture to a reactor to yield a product mixture, wherein the reactor comprises an oxidative coupling of methane (OCM) catalyst, wherein the reactant mixture comprises methane, oxygen, and a chlorine radical precursor, wherein the chlorine radical precursor is present in the reactant mixture in an amount of from about 0.5 vol. % to about 3 vol. %, based on the total volume of the reactant mixture, wherein the product mixture comprises ethylene, ethane, and unreacted methane, wherein the OCM catalyst comprises (1) a redox agent in an amount of from about 1 wt. % to about 25 wt. %, based on the total weight of the OCM catalyst, and (2) an alkali metal, an alkaline earth metal, or both, in an amount of less than about 3 wt. %, based on the total weight of the OCM catalyst; and
(b) recovering at least a portion of the ethylene from the product mixture.

18. The process of claim 17, wherein the chlorine radical precursor comprises hydrogen chloride (HCl); and wherein the OCM catalyst comprises (1) manganese (Mn) in an amount of from about 10 wt. % to about 20 wt. %, based on the total weight of the OCM catalyst, (2) sodium (Na), calcium (Ca), or both in an amount of from about 1 wt. % to about 3 wt. %, based on the total weight of the OCM catalyst, and (3) a SiO₂ support.

19. A process for producing ethylene comprising:
(a) continuously feeding a reactant mixture and a chlorine radical precursor for an activation time period to a reactor comprising an oxidative coupling of methane (OCM) catalyst to activate the OCM catalyst and to yield a first product mixture, wherein the chlorine radical precursor is introduced to the reactor in an amount of from about 2 vol. % to about 5 vol. %, based on the total volume of the reactant mixture, wherein the reactant mixture comprises methane and oxygen, wherein the first product mixture comprises ethylene, ethane, and unreacted methane, wherein the OCM catalyst comprises (1) a redox agent in an amount of from about 1 wt. % to about 25 wt. %, based on the total weight of the OCM catalyst, and (2) an alkali metal, an alkaline earth metal, or both, in an amount of equal to or greater than about 3 wt. %, based on the total weight of the OCM catalyst;
(b) discontinuing the introduction of the chlorine radical precursor to the reactor while continuing to feed the reactant mixture to the reactor for a reaction time period to produce a second product mixture, wherein the second product mixture comprises ethylene, ethane, and unreacted methane;
(c) repeating steps (a) and (b) as necessary to achieve a target methane conversion and/or a target ethylene selectivity; and
(d) recovering at least a portion of the ethylene from the first product mixture and/or the second product mixture; wherein the first product mixture and/or the second product mixture are characterized by an ethylene to ethane molar ratio of equal to or greater than about 8:1.

20. The process of claim 19, wherein the chlorine radical precursor comprises hydrogen chloride (HCl); and wherein the OCM catalyst comprises (1) manganese (Mn) in an amount of from about 15 wt. % to about 25 wt. %, based on the total weight of the OCM catalyst, (2) sodium (Na), calcium (Ca), or both in an amount of from about 10 wt. % to about 20 wt. L%, based on the total weight of the OCM catalyst, and (3) a SiO₂ support.
